(19) United States
(12) Patent Application Publication
(10) Pub. No.: US 2018/0003724 A1
(43) Pub. Date: Jan. 4, 2018

(54) ALZHEIMER'S DISEASE DIAGNOSTIC PANELS AND METHODS FOR THEIR USE

(71) Applicant: Integrated Diagnostics, Inc., Seattle, WA (US)

(72) Inventors: Xiao-Jun LI, Bellevue, WA (US); Paul Edward KEARNEY, Seattle, WA (US)

(21) Appl. No.: 15/685,535

(22) Filed: Aug. 24, 2017

Related U.S. Application Data
(63) Continuation of application No. 14/672,908, filed on Mar. 30, 2015, now abandoned.
Application No. 14/672,908 is a continuation of application No. 13/306,858, filed on Nov. 29, 2011, now abandoned.
(60) Provisional application No. 61/417,871, filed on Nov. 29, 2010.

(51) Int. Cl. G01N 33/68

(52) U.S. Cl. CPC G01N 33/6896; G01N 2800/60; G01N 2800/2821; G01N 2333/916; G01N 2333/4703; G01N 2333/811; G01N 2333/775; G01N 2333/91085

(57) ABSTRACT

Novel compositions, methods, assays and kits directed to a diagnostic panel for Alzheimer's disease are provided. In one embodiment, the diagnostic panel includes one or more proteins associated with Alzheimer's disease.

### CROSS-REFERENCE TO RELATED APPLICATIONS

This application is a continuation of U.S. patent application Ser. No. 14/672,908, filed on Mar. 30, 2015, which is a continuation of U.S. patent application Ser. No. 13/306,858, filed on Nov. 29, 2011, which claims priority benefit of U.S. Provisional Patent Application No. 61/417,871, filed on Nov. 29, 2010, the contents of each of which are incorporated herein by reference in their entireties.

### INCORPORATION-BY-REFERENCE OF SEQUENCE LISTING

The contents of the text file named “IDIA_003_C01_Sequence_Listing_ST25.txt”, which was created on Mar. 26, 2015 and is 3 KB in size, are hereby incorporated by reference in their entireties.

### BACKGROUND

One aim of modern diagnostic medicine is to better identify sensitive diagnostic methods to determine changes in health status. A variety of diagnostic assays and computational methods are used to monitor health. Improved sensitivity is an important goal of diagnostic medicine. Early diagnosis and identification of disease and changes in health status may permit earlier intervention and treatment that will produce healthier and more successful outcomes for the patient. Diagnostic markers are important for prognosis, diagnosis and monitoring disease and changes in health status. In addition, diagnostic markers are important for predicting response to treatment and selecting appropriate treatment and monitoring response to treatment.

Many diagnostic markers are identified in the blood. However, identification of appropriate diagnostic markers is challenging due to the number, complexity and variety of proteins in the blood. Distinguishing between high abundance and low abundance detectable markers requires novel methods and assays to determine the differences between normal levels of detectable markers and changes of such detectable markers that are indicative of changes in health status. The present invention provides novel compositions, methods and assays to fulfill these and other needs.

### SUMMARY

In one embodiment, a diagnostic Alzheimer's disease panel is provided. The diagnostic Alzheimer's disease panel may include one or more proteins associated with Alzheimer's disease. In one embodiment, the one or more proteins associated with Alzheimer's disease may be selected from A1BG, APOA4, APOD, ARSA, ATP2A2, BDNF, CACNB2, CALML3, CDH5, CLU, COL18A1, COL1A2, CPN1, CSF1R, EPB41, EPHA8, F13A1, GALR3, GC, GNAQ, GPR113, GRIN2A, GRN, GSN, HPX, INADL, ITIH1, ITIH2, Kng1, LAMB2, LRP8, LTBP1, MMP16, MPDZ, MTOR, NMB, NTRK2, PACSIN1, PARD3, PKDREJ, PON1, PTPRB, SEMG1, SERPINA3, SERPINA4, SERPINF1, SNCB, SYTL4, TMPRSS2 and VTN. In another embodiment, the one or more proteins associated with Alzheimer's disease may be selected from F13A1, PON1, ITIH1, CLU, APOD, GSN and APOA4.

In another embodiment, the diagnostic Alzheimer's disease panel is a set of seven proteins that includes F13A1, PON1, ITIH1, CLU, APOD, GSN and APOA4. In another embodiment, the diagnostic Alzheimer's disease panel is a set of three proteins that includes GSN, F13A1 and PON1.

In another embodiment, a diagnosis of Alzheimer's disease may be made based on the detection of differential expression or differential presence of four or more significant transitions that are associated with the Alzheimer's disease panel. The Alzheimer's disease diagnosis may be a determination of whether a patient is experiencing the early stages of the disease.

### BRIEF DESCRIPTION OF THE DRAWINGS

FIG. 1 are representative images of a brain with diagnosed Alzheimer's disease having substantial loss of brain tissue (left) as compared to a normally aged brain in a normal elderly control (NEC) (right).

FIG. 2 is a graph showing the delay in a patient's decline in quality of life as a result of earlier diagnosis and treatment of Alzheimer's disease.

FIG. 3 is a graph showing the delay in admission to long-term care and shorter stays in such facilities as a result of early diagnosis and treatment of Alzheimer's disease.

FIG. 4 is a regression plot illustrating the correlation of the blood protein biomarkers described herein to mini mental state evaluation (MMSE) score (r²=0.75, p<0.0022).

FIG. 5 is a schematic illustrating MRM technology related to the selected peptides and transitions for a target protein, Protein X.

FIG. 6 is a schematic diagram illustrating selected peptides and transitions for three target proteins, Protein X, Y and Z.

FIG. 7 is a set of bar graphs illustrating the intensity of F13A1 significant transitions SEQ ID NO: 1 (LIASMSSDSLR (590.3-1066.3) (A)), SEQ ID NO: 1 (LIASMSSDSLR (590.3-953.2) (B)), SEQ ID NO: 2 (STVLTIPEIIIK, transition 1 (C)) and SEQ ID NO: 2 (STVLTIPEIIIK, transition 2 (D)) in untreated Alzheimer's disease (DATU) blood plasma samples as compared to normal elderly control (NEC) samples (+p<0.05).

FIG. 8 is a series of receiver operating characteristic (ROC) curves illustrating the diagnostic performance for each of the following 14 individual significant transitions: SEQ ID NO: 1 (LIASMSSDSLR (590.3-1066.3) (AUC=0.73)), SEQ ID NO: 3 (IQNILTEEPK (592.8-829.4) (AUC=0.72)), SEQ ID NO: 1 (LIASMSSDSLR (590.3-953.2) (AUC=0.71)), SEQ ID NO: 3 (IQNILTEEPK (592.8-943.4) (AUC=0.70)), SEQ ID NO: 4 (GSLVQASEANLQAAQDFVR (1002.5-1448.6) (AUC=0.66)), SEQ ID NO: 5 (EIQNAVNGVK (536.3-417.2) (AUC=0.66)), SEQ ID NO 7 (VLNQELR (436.2-659.3) (AUC=0.63)), SEQ ID NO: 4 (GSLVQASEANLQAAQDFVR (1002.5-1232.6) (AUC=0.64)), SEQ ID NO: 7 (TGAQELLR (444.2-530.3) (AUC=0.65)), SEQ ID NO: 8 (ALVQQMEQLR (608.3-932.5) (AUC=0.67)), SEQ ID NO: 7 (TGAQELLR (444.2-658.4) (AUC=0.64)), SEQ ID NO: 9 (ELDESLQVAER (644.8-802.4) (AUC=0.66)), SEQ ID NO: 6 (VLNQELR (436.2-772.4) (AUC=0.61)) and SEQ ID NO: 10 (EVAFDLEIPK (580.8-861.5) (AUC=0.66)).

FIG. 9 is a receiver operating characteristic (ROC) curve for illustrating the diagnostic performance of the multivariate Alzheimer's disease panel (AUC=0.82) as determined by the significant transitions listed in FIG. 8.

FIG. 10 is a receiver operating characteristic (ROC) curve for illustrating the diagnostic performance of the 8 individual significant transitions for four peptides (SEQ ID NO: 7 (TGAQELLR), SEQ ID NO: 1 (LIASMSSDSLR), SEQ ID NO: 3 (IQNILTEEPK) and SEQ ID NO: 2 (STVLTIPEIIIK); two transitions per peptide) and a receiver operating characteristic (ROC) curve for illustrating the diagnostic performance of a 3-protein Alzheimer's disease panel (GSN, F13A1 and PON1; AUC=0.80) based on the combined performance of the 8 individual significant transitions.

FIG. 11 is a bar graph that shows the estimated limit of quantification (LOQ) of the most intense peptide for each of a set of 50 target Alzheimer's disease related proteins: A1BG, APOA4, APOD, ARSA, ATP2A2, BDNF, CACNB2, CALML3, CDH5, CLU, COL18A1, COL1A2, CPN1, CSF1R, EPB41, EPHA8, F13A1, GALR3, GC, GNAQ, GPR113, GRIN2A, GRN, GSN, HPX, INADL, ITIH1, ITIH2, Kng1, LAMB2, LRP8, LTBP1, MMP16, MPDZ, MTOR, NMB, NTRK2, PACSIN1, PARD3, PKDREJ, PON1, PTPRB, SEMG1, SERPINA3, SERPINA4, SERPINF1, SNCB, SYTL4, TMPRSS2 and VTN. The graph illustrates that the target proteins can be detected at concentrations in the ng/mL range.

### DETAILED DESCRIPTION

The present disclosure provides novel compositions, methods, assays and kits directed to a diagnostic panel for Alzheimer's disease panel. In one embodiment, the diagnostic panel includes one or more proteins associated with Alzheimer's disease. The diagnostic panel can be used for prognosis and diagnosis, monitoring treatment and monitoring response to treatment.

According to some embodiments, the one or more proteins associated with Alzheimer's disease may be selected from alpha-1-B glycoprotein (A1BG), apolipoprotein A4 (APOA4), apolipoprotein D (APOD), arylsulfatase A (ARSA), sarco(endo)plasmic reticulum calcium-ATPase 2 (ATP2A2), brain-derived neurotrophic factor (BDNF), voltage-dependent L-type calcium channel subunit beta-2 (CACNB2), calmodulin-like protein 3 (CALML3), cadherin 5, type 2 (CDH5), clusterin (CLU), collagen alpha-1(XVIII) chain (COL18A1), collagen alpha-2(I) chain (COL1A2), carboxypeptidase N catalytic chain (CPN1), colony stimulating factor 1 receptor (CSF1R), erythrocyte membrane protein band 4.1 (EPB41), ephrin type-A receptor 8 (EPHA8), coagulation factor XIII A chain (F13A1), galanin receptor 3 (GALR3), gc-globulin (GC), guanine nucleotide-binding protein G(q) subunit alpha (GNAQ), probable G-protein coupled receptor 113 (GPR113), glutamate [NMDA] receptor subunit epsilon-1 (GRIN2A), granulin (GRN), gelsolin (GSN), hemopexin (HPX), inaD-like protein (INADL), inter-alpha-trypsin inhibitor heavy chain H1 (ITIH1), inter-alpha-trypsin inhibitor heavy chain H2 (ITIH2), High-molecular-weight kininogen (Kng1), laminin subunit beta-2 (LAMB2), low-density lipoprotein receptor-related protein 8 (LRP8), latent TGF-beta binding protein 1 (LTBP1), matrix metalloproteinase 16 (MMP16), multiple PDZ domain protein (MPDZ), mammalian target of rapamycin (MTOR), neuromedin B (NMB), neurotrophic tyrosine kinase receptor 2 (NTRK2), protein kinase C and casein kinase substrate in neurons protein 1 (PACSIN1), partitioning defective 3 homolog (PARD3), polycystic kidney disease (polycystin) and REJ homolog (sperm receptor for egg jelly homolog, sea urchin) (PKDREJ), paraoxonase 1 (PON1), receptor-type tyrosine-protein phosphatase beta (PTPRB), semenogelin-1 (SEMG1), alpha 1-antichymotrypsin (SERPINA3), kallistatin (SERPINA4), serpin F1 (SERPINF1), beta-synuclein (SNCB), synaptotagmin-like protein 4 (SYTL4), transmembrane protease, serine 2 (TMPRSS2) and vitronectin (VTN).

In other embodiments, the one or more proteins associated with lung cancer may be selected from coagulation factor XIIIa (F13A1), paraoxonase 1 (PON1), inter-alpha-trypsin inhibitor heavy chain H1 (ITIH1), clusterin (CLU), apolipoprotein D (APOD), gelsolin (GSN) and apolipoprotein A4 (APOA4).

In another embodiment, the diagnostic Alzheimer's disease panel is a set of seven proteins that includes F13A1, PON1, ITIH1, CLU, APOD, GSN and APOA4. In yet another embodiment, the diagnostic Alzheimer's disease panel is a set of three proteins: coagulation factor XIIIa (F13A1), paraoxonase 1 (PON1) and gelsolin (GSN). The Alzheimer's disease panels identified herein are sensitive and accurate diagnostic tools that can be measured in a biological sample. The Alzheimer's disease panels include a group or set of Alzheimer's disease-specific proteins that have been associated with the disease and have been detected in biological samples of subjects who have Alzheimer's disease and normal control populations.

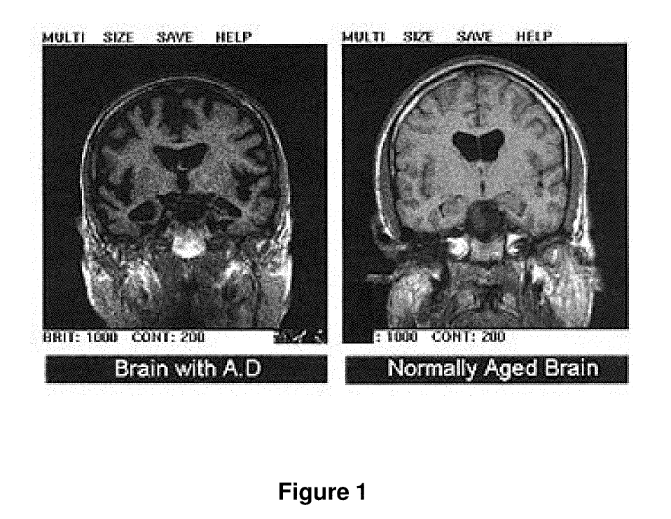

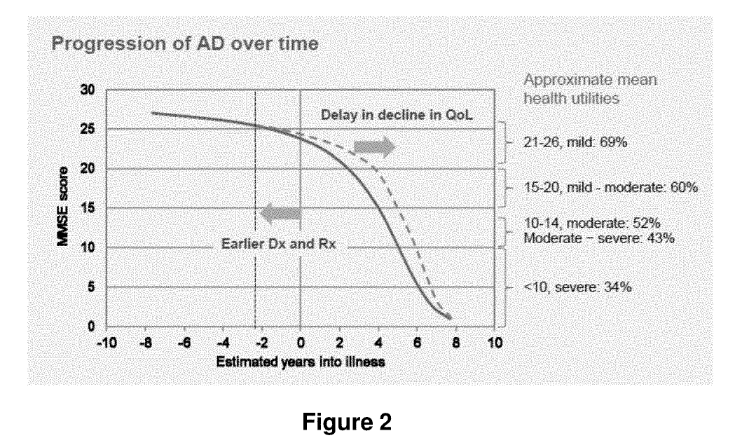

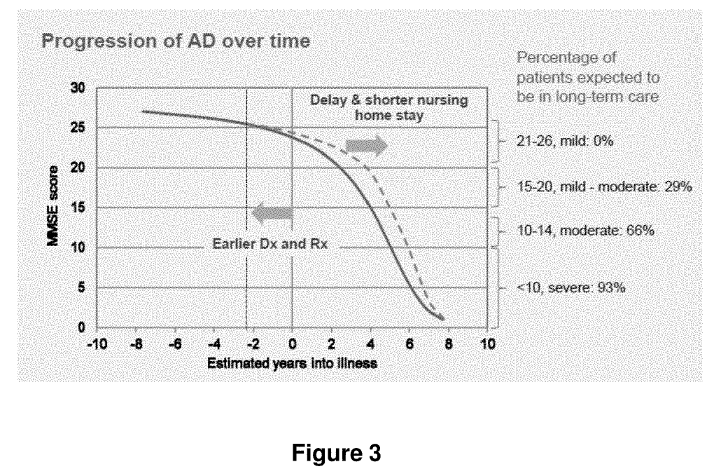

The diagnostic panels of the present disclosure can be used for diagnosing Alzheimer's disease in a subject. As used herein, the term “subject” refers to any animal (e.g., a mammal), including but not limited to humans, non-human primates, rodents, dogs, pigs, and the like. In one aspect, the Alzheimer's disease panels may be used to diagnose Alzheimer's disease before the disease is too far advanced for intervention (see FIG. 1). Currently, early diagnosis of Alzheimer's disease is based on a patient exhibiting minimal cognitive impairment (MCI) and ruling out other central nervous system neuropathies, however, there are no established diagnostic tools or universal standards for classifying early stages of the disease. Early intervention in the development of Alzheimer's disease can delay a patient's decline in quality of life (FIG. 2) and can delay admission to long-term care and shorten stays in such facilities (FIG. 3).

In one embodiment, a method for diagnosing Alzheimer's disease includes obtaining a biological sample (e.g., a blood, plasma or serum sample) from a subject having or suspected of having a form of cognitive impairment or dementia and determining whether a differential expression or differential presence of one or more proteins, peptides or transitions associated with the Alzheimer's disease panels described herein. Such a method may further include a system for distinguishing Alzheimer's disease from other forms of dementia or cognitive impairment to allow early detection of Alzheimer's disease and risk factors. For example, methods described herein may be used to classify or distinguish between Alzheimer's disease from a normal aging effect on cognitive function (i.e., diseased patients as compared to normal elderly controls, (NEC)), Untreated Alzheimer's disease (UTAD), as compared to Treated Alzheimer's Disease (TTAD), Alzheimer's disease as compared to mild cognitive impairment, and additional comparisons between other stages of cognitive disorders.

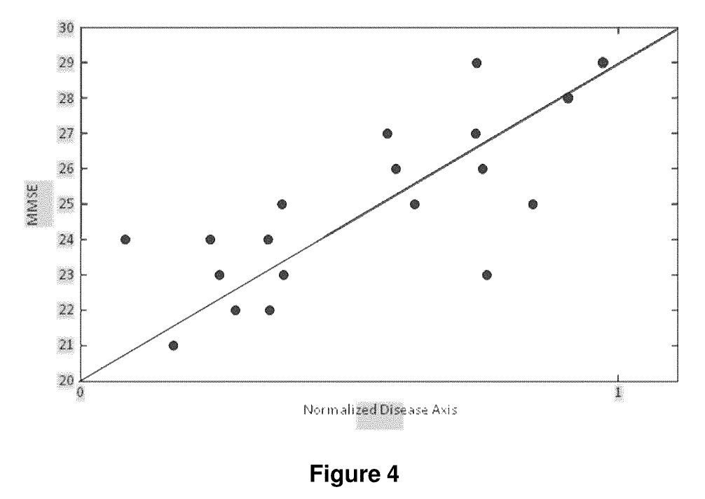

In some embodiments, the method for diagnosing Alzheimer's disease as described above may optionally include administration of a mini mental state examination (MMSE) for validation of a diagnosis made based on the Alzheimer's disease panels. An MSE is a questionnaire that tests for cognitive impairment and is often used to screen for dementia. An MMSE, when used in combination with the methods described herein, may be used to validate the results of the methods for diagnosing Alzheimer's disease based on the Alzheimer's disease panels described herein. As shown in FIG. 4, the biomarkers associated with the Alzheimer's disease panels are correlated to the MMSE scores.

The diagnostic Alzheimer's disease panels used in the methods described herein may be used to diagnose Alzheimer's disease and may be used to distinguish the development of Alzheimer's disease from less severe forms of dementia or may by used to rule out other forms of cognitive impairment or dementia. Examples of cognitive disorders or dementia that may be ruled out by the methods that use the Alzheimer's disease panels described herein include, include, but are not limited to normal aging, Parkinson's disease, vascular dementia, dementia with Lewy bodies, progressive supranuclear palsy, corticobasal degeneration, frontotemporal lobular degeneration and Bechet's disease.

According to the methods described herein, a diagnosis of Alzheimer's disease may be made based on the detection of one or more proteins, peptides or transitions that are differentially present or differentially expressed in a biological sample (e.g., blood, plasma or serum). In one embodiment, the one or more peptides or transitions are associated with the proteins of the Alzheimer's disease panels (i.e., F13A1, PON1, ITIH1, CLU, APOD, GSN and APOA4).

In one embodiment, a diagnosis of Alzheimer's disease may be made based on the detection of one or more significant transitions in a biological sample (e.g., blood, plasma or serum). In one aspect the one or more significant transitions are selected from SEQ ID NO: 1 (LIASMSSDSLR (590.3-1066.3)), SEQ ID NO: 1 (LIASMSSDSLR (590.3-953.2)), SEQ ID NO: 4 (GSLVQASEANLQAAQDFVR (1002.5-1448.6)), SEQ ID NO: 4 (GSLVQASEANLQAAQDFVR (1002.5-1232.6)), SEQ ID NO: 3 (IQNILTEEPK (592.8-829.4)), SEQ ID NO: 3 (IQNILTEEPK (592.8-943.4)), SEQ ID NO: 5 (EIQNAVNGVK (536.3-417.2)), SEQ ID NO: 7 (TGAQELLR (444.2-530.3)), SEQ ID NO: 7 (TGAQELLR (444.2-658.4)), SEQ ID NO: 6 (VLNQELR (436.2-659.3)), SEQ ID NO: 6 (VLNQELR (436.2-772.4)), SEQ ID NO: 8 (ALVQQMEQLR (608.3-932.5)), SEQ ID NO: 9 (ELDESLQVAER (644.8-802.4)), SEQ ID NO:11 (EVAFDLEIPK (580.8-861.5)).

The phrase “differentially present” or “differentially expressed” refers to different in the quantity or intensity of a marker present in a sample taken from patients having Alzheimer's disease as compared to a comparable sample taken from patients who do not have Alzheimer's disease. For example, a protein, polypeptide or peptide is differentially expressed between the samples if the amount of the protein, polypeptide or peptide in one sample is significantly different (i.e., p<0.05) from the amount of the protein, polypeptide or peptide in the other sample. Further, a peptide ion transition (a “transition,” described below) is differentially present between the samples if the intensity of the transition is significantly different (i.e., p<0.05) from the intensity of the transition in the other sample. It should be noted that if the protein, polypeptide, transition or other marker is detectable in one sample and not detectable in the other, then such a marker can be considered to be differentially present.

To increase the sensitivity of protein detection, a blood, plasma or serum sample may be initially processed to by any suitable method known in the art. In one embodiment, blood proteins may be initially processed by a glycocapture method, which enriches for glycosylated proteins, allowing quantification assays to detect proteins in the high pg/ml to low ng/ml concentration range. Example methods of glycocapture are described in detail in U.S. Pat. No. 7,183,188, issued Jun. 3, 2003; U.S. Patent Application Publication No. 2007/0099251, published May 3, 2007; U.S. Patent Application Publication No. 2007/0202539, published Aug. 30, 2007; U.S. Patent Application Publication No. 2007/0269895, published Nov. 22, 2007; and U.S. Patent Application Publication No. 2010/0279382, published Nov. 4, 2010, all of which are hereby incorporated by reference in their entirety, as if fully set forth herein. In another embodiment, blood proteins may be initially processed by a protein depletion method, which allows for detection of commonly obscured biomarkers in samples by removing abundant proteins. In one embodiment, the protein depletion method is a GenWay depletion method.

Differential expression or differential presence of the proteins of the protein panels may be measured and/or quantified by any suitable method known in the art including, but not limited to, reverse transcriptase-polymerase chain reaction (RT-PCR) methods, microarray, serial analysis of gene expression (SAGE), gene expression analysis by massively parallel signature sequencing (MPSS), immunoassays such as ELISA, immunohistochemistry (IHC), mass spectrometry (MS) methods, transcriptomics and proteomics. With respect to mass spectrometry, the most common modes of acquiring LC/MS data are: (1) Full scan acquisition resulting in the typical total ion current plot (TIC), (2) Selected Ion Monitoring (SIM) or (3) multiple reaction monitoring (MRM).

In one embodiment, differential expression or differential presence of the proteins of the panel is quantified by a mass spectrometry method. The use of mass spectrometry, in accordance with the disclosed methods and Alzheimer's disease specific panels provides information on not only the mass to charge ratio (m/z ratio) of ions generated from a sample and the relative abundance of such ions. Under standardized experimental conditions, the abundance of a noncovalent biomolecule-ligand complex ion with the ion abundance of the noncovalent complex formed between a biomolecule and a standard molecule, such as a known substrate or inhibitor is compared. Through this comparison, binding affinity of the ligand for the biomolecule, relative to the known binding of a standard molecule and the absolute binding affinity may be determined.

A variety of mass spectrometry systems can be employed for identifying and/or quantifying Alzheimer's disease biomarkers or Alzheimer's disease biomarker panels in biological samples. In some embodiments, analytes may be quantified by liquid chromatography-mass spectrometry (LC-MS) using eXtracted Ion Chromatograms (XIC). Data are collected in full MS scan mode and processed post-acquisition, to reconstruct the elution profile for the ion(s) of interest, with a given m/z value and a tolerance. XIC peak heights or peak areas are used to determine the analyte abundance.

In other embodiments, quantification of analytes is achieved by selected ion monitoring (SIM) performed on scanning mass spectrometers, by restricting the acquisition mass range around the m/z value of the ion(s) of interest. The narrower the mass range, the more specific the SIM assay. SIM experiments are more sensitive than XICs from full scans because the MS is allowed to dwell for a longer time over a small mass range of interest. Several ions within a given m/z range can be observed without any discrimination and cumulatively quantified; quantification is still performed using ion chromatograms.

In other embodiments, selected reaction monitoring (SRM) is used. SRM exploits the capabilities of triple quadrupole (QQQ) MS for quantitative analysis of an analyte. SRM is a non-scanning technique, generally performed on triple quadrupole (QQQ) instruments in which fragmentation is used as a means to increase selectivity. In SRM, the first and the third quadrupoles act as filters to specifically select predefined m/z values corresponding to the peptide ion and a specific fragment ion of the peptide, whereas the second quadrupole serves as collision cell. In SRM experiments, two mass analyzers are used as static mass filters, to monitor a particular fragment ion of a selected precursor ion. The selectivity resulting from the two filtering stages combined with the high-duty cycle results in quantitative analyses with unmatched sensitivity. The specific pair of m/z values associated with the precursor and fragment ions selected is referred to as a ‘transition’ (e.g., 673.5/534.3). Several such transitions (precursor/fragment ion pairs) are monitored over time, yielding a set of chromatographic traces with the retention time and signal intensity for a specific transition as coordinates.

Multiplexed SRM transitions can be measured within the same experiment on the chromatographic time scale by rapidly cycling through a series of different transitions and recording the signal of each transition as a function of elution time. The method, also referred to as multiple reaction monitoring mass spectrometry (MRM), allows for additional selectivity by monitoring the chromatographic co-elution of multiple transitions for a given analyte.

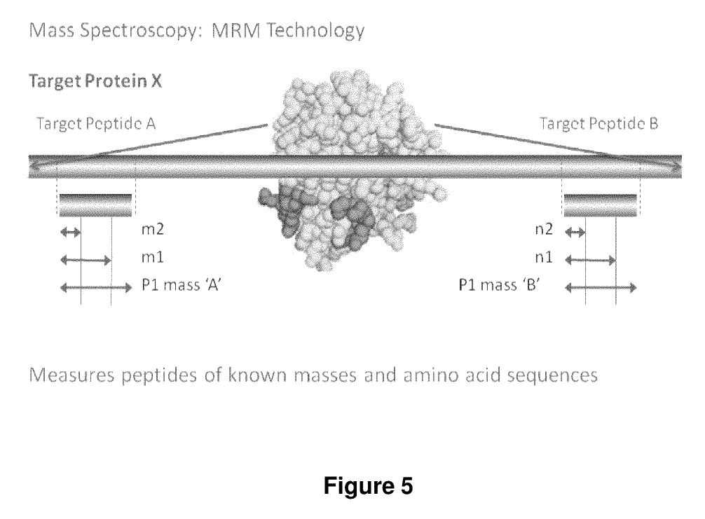

In some embodiments, an MRM-triggered MS/MS (MRM-MS/MS) method was used to develop an MRM assay for selection and quantification of target proteins associated with Alzheimer's disease. For each target protein, several peptides were selected based on previous identification or presence in the public peptide MS/MS spectra databases TheGPM, PeptideAtlas and HUPO. The MRM-MS/MS method was developed by calculating for each peptide the precursor mass of the doubly and triply charged peptide ions and the first y fragment ion with an m/z greater than m/z (precursor)+20 Da. If these calculated transitions were observed during the MRM scan, a full MS/MS spectrum of the precursor peptide ion was acquired. The two most intense b or y fragments in the MS/MS spectrum for each peptide were recorded. Then, the two most suitable peptides for the MRM assay were selected based on observed signal intensity and origin of the peptide. FIG. 5 is an illustration of selected peptides (Target Peptide A, Target Peptide B) having known masses (P1 mass ‘A’ and P1 mass ‘B’) and transitions (m1, m2, n1, n2) for a target protein X.

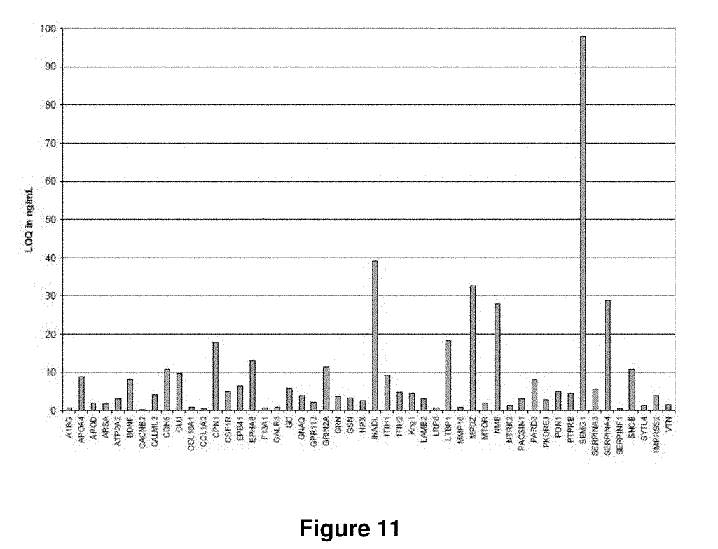

Based on the peptide and transition selection described above, the MRM assay used in accordance with the methods for diagnosing Alzheimer's disease described herein measures the intensity of the four transitions that correspond to the selected peptides associated with each targeted protein. The achievable limit of quantification (LOQ) may be estimated for each peptide according to the observed signal intensities during this analysis. For example, for a set of target proteins associated with Alzheimer's disease (A1BG, APOA4, APOD, ARSA, ATP2A2, BDNF, CACNB2, CALML3, CDH5, CLU, COL18A1, COL1A2, CPN1, CSF1R, EPB41, EPHA8, F13A1, GALR3, GC, GNAQ, GPR113, GRIN2A, GRN, GSN, HPX, INADL, ITIH1, ITIH2, Kng1, LAMB2, LRP8, LTBP1, MMP16, MPDZ, MTOR, NMB, NTRK2, PACSIN1, PARD3, PKDREJ, PON1, PTPRB, SEMG1, SERPINA3, SERPINA4, SERPINF1, SNCB, SYTL4, TMPRSS2 and VTN), the estimated LOQ for the most intense peptide for each Alzheimer's disease-related protein is shown in FIG. 11.

The intensity for each of the four transitions associated with the Alzheimer's disease panels are measured by MRM assay and compared between a cohort of Alzheimer's disease patient samples and a cohort of control patient samples. A control patient may be an individual who has cognitive impairment due to the normal effects of aging or who has no cognitive impairment. An individual transition intensity in the cohort of Alzheimer's disease patient samples that is significantly different than the corresponding individual transition intensity in the cohort of control patient samples is selected as a significant transition biomarker. The protein that corresponds to the significant transition biomarker is designated as a protein in an Alzheimer's disease panel.

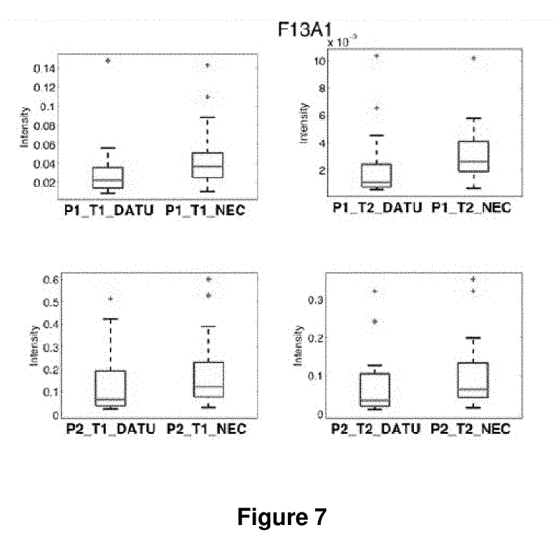

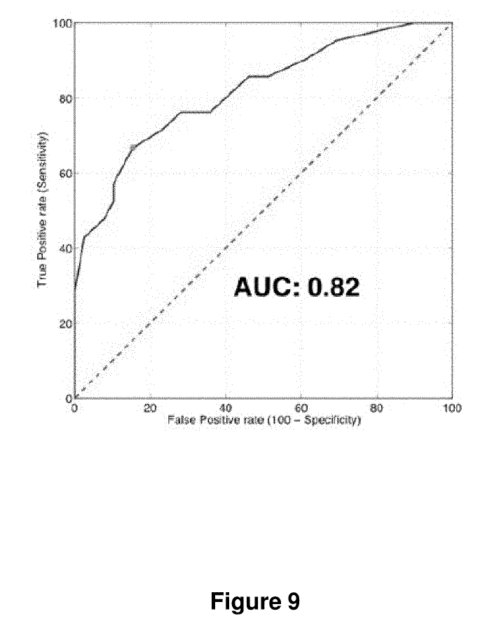

To determine their diagnostic performance, a receiver operating characteristic (ROC) curve was generated for each significant transition biomarker identified above. A “receiver operating characteristic (ROC) curve” is a generalization of the set of potential combinations of sensitivity and specificity possible for predictors. A ROC curve is a plot of the true positive rate (sensitivity) against the false positive rate (1-specificity) for the different possible cut-points of a diagnostic test. FIGS. 7 and 9 are a graphical representation of the functional relationship between the distribution of a biomarker's or a panel of biomarkers' sensitivity and specificity values in a cohort of diseased subjects and in a cohort of non-diseased subjects. The area under the curve (AUC) is an overall indication of the diagnostic accuracy of (1) a biomarker or a panel of biomarkers and (2) a receiver operating characteristic (ROC) curve. AUC is determined by the “trapezoidal rule.” For a given curve, the data points are connected by straight line segments, perpendiculars are erected from the abscissa to each data point, and the sum of the areas of the triangles and trapezoids so constructed is computed.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. Further, all references cited above and in the examples below are hereby incorporated by reference in their entirety, as if fully set forth herein.

### Example 1: Generation and Performance of an Alzheimer's Disease Panel

Sample Processing.

A set of 130 blood plasma samples were obtained from a cohort of untreated Alzheimer's disease patients (“the DATU samples;” n=21), a cohort of Alzheimer's disease patients that were treated with donepezil/Aricept® (“the DATT samples;” n=31), a cohort of patients with mild cognitive impairment (“the MDI samples;” n=39) and a cohort of normal elderly control patients that represent a normal aging brain (“the NEC samples;” n=39). In addition, 11 tissue test samples were obtained from neurosurgical controls (“the NC samples;” n=10) and from subjects with Alzheimer's disease (“the NJ samples;” n=1). Neurosurgical controls were obtained from patients undergoing neurosurgical treatment for deep seated tumors, for which removal of apparently normal tissue was a necessary part of the surgical procedure. The samples were initially processed by a GenWay depletion method as described above. The enriched target proteins were then subjected to an MRM as discussed below.

MRM: Selection of Transition Biomarkers and Corresponding Alzheimer's Disease Panel.

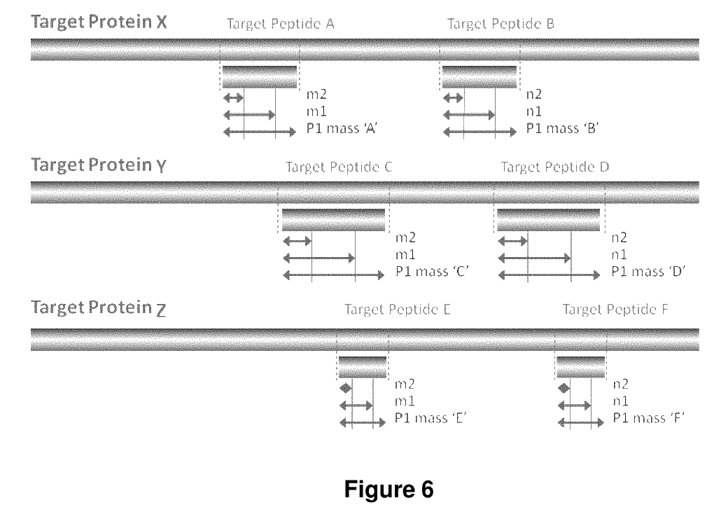

An MRM assay measures 1-2 target peptides with known masses and amino acid sequences (see FIG. 6, Target Peptide A, Target Peptide B, Target Peptide C, Target Peptide D, Target peptide E, Target Peptide F) for each target protein. The MRM device then searches for the known peptide masses (see FIG. 6, P1 mass ‘A,’ P1 mass ‘B,’ P1 mass ‘C,’ P1 mass ‘D,’ P1 mass ‘E,’ P1 mass ‘F’). When a peptide with the known peptide mass is detected, the peptide is fragmented. The MRM device measures the intensity of 2 fragments per peptide, (aka, two transitions per peptide). Thus the results of the MRM assay typically results in an average of 2-4 transition intensity measurements per protein (see FIG. 6, m1, m2, n1, n2).

A panel of 50 proteins was targeted by an MRM assay as described above. From these 50 target proteins, 100 peptides and 200 transitions were selected (each peptide had two transitions). Three replicate MRM analyses were performed to detect presence or expression of the proteins corresponding to the transitions. A high ranking protein approach was used to determine the diagnostic importance of the detected proteins based on discovery studies and biomarkers cited in the literature (see Pubmed associations and representative references in Table 1, below).

The intensities of each transition were compared between the Alzheimer's disease samples and the control samples (Mann-Whitney U-test). For each target protein, the two transitions having the highest intensity were compared to determine if the target protein distinguished diseased samples from normal samples or normal aged samples from the aging brain. Specifically, the two highest transition intensity measurements for each target protein in the Alzheimer's disease samples were compared to the two highest transition intensity measurements for each target protein in the control samples. A transition was considered to be significant if the p value was less than 0.05. Fourteen transitions were found to be significant between Alzheimer's disease and control samples, corresponding to 7 protein biomarkers. Table 1, shows the biomarker proteins identified. Examples of significant transition intensity determinations are shown in FIG. 7 (which corresponds to F13A1 transitions SEQ ID NO: 1 (LIASMSSDSLR (590.3-1066.3) (A)), SEQ ID NO: 1 (LIASMSSDSLR (590.3-953.2) (B)), SEQ ID NO: 2 (STVLTIPEIIIK, transition 1 (C)) and SEQ ID NO: 2 (STVLTIPEIIIK, transition 2 (D)).

TABLE 1
Biomarkers identified using median of all replicates.

| | No. of | No. of | No. of | |
|---|---|---|---|---|
| | Significant | Significant | Pubmed | |
| Protein | Transitions | Peptides | Associations | Representative Reference |
| F13A1 | 2 | 1 | 5 | Immunohistochemical detection of |
| | | | | coagulation factor XIIIa in postmortem |
| | | | | human brain tissue. |
| PON1 | 2 | 1 | 21 | Association study of the paraoxonase 1 |
| | | | | gene with the risk of developing |
| | | | | Alzheimer's disease. |
| ITIH1 | 3 | 2 | 7 | |
| CLU | 2 | 2 | 113 | Alzheimer disease: Plasma clusterin |
| | | | | predicts degree of pathogenesis in AD. |
| APOD | 2 | 1 | 20 | Increased levels of apolipoprotein D in |
| | | | | cerebrospinal fluid and hippocampus of |
| | | | | Alzheimer's patients. |
| GSN | 2 | 1 | 49 | Plasma gelsolin is decreased and |
| | | | | correlates with rate of decline in |
| | | | | Alzheimer's disease. |
| APOA4 | 1 | 1 | 2 | |

Significant Transition Diagnostic Performance.

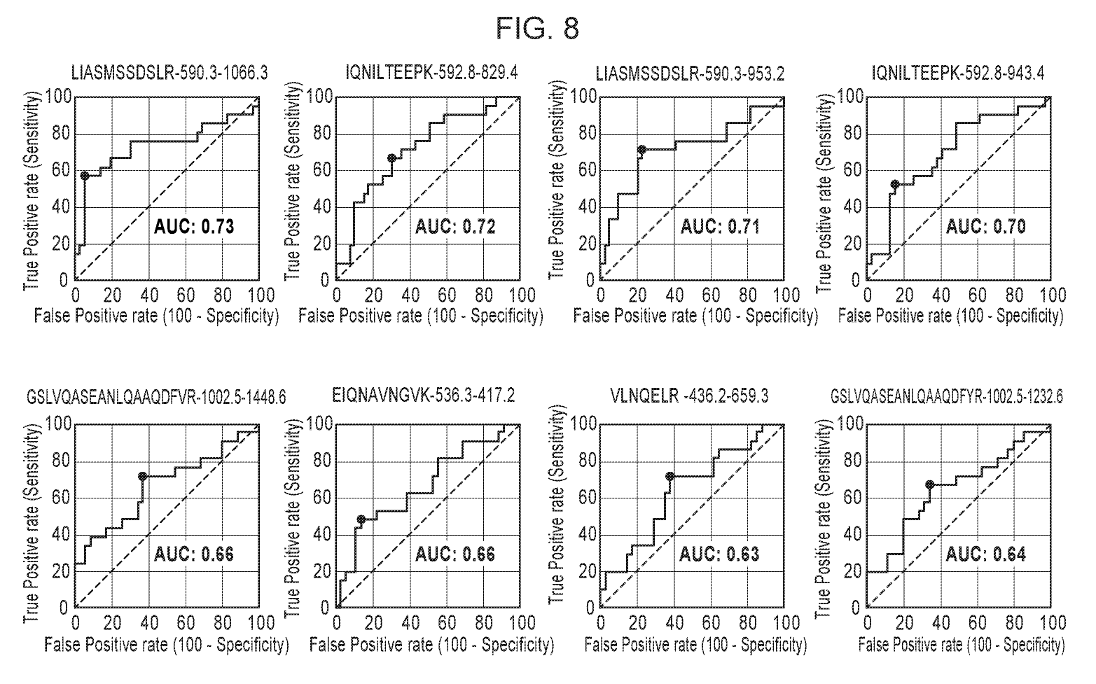

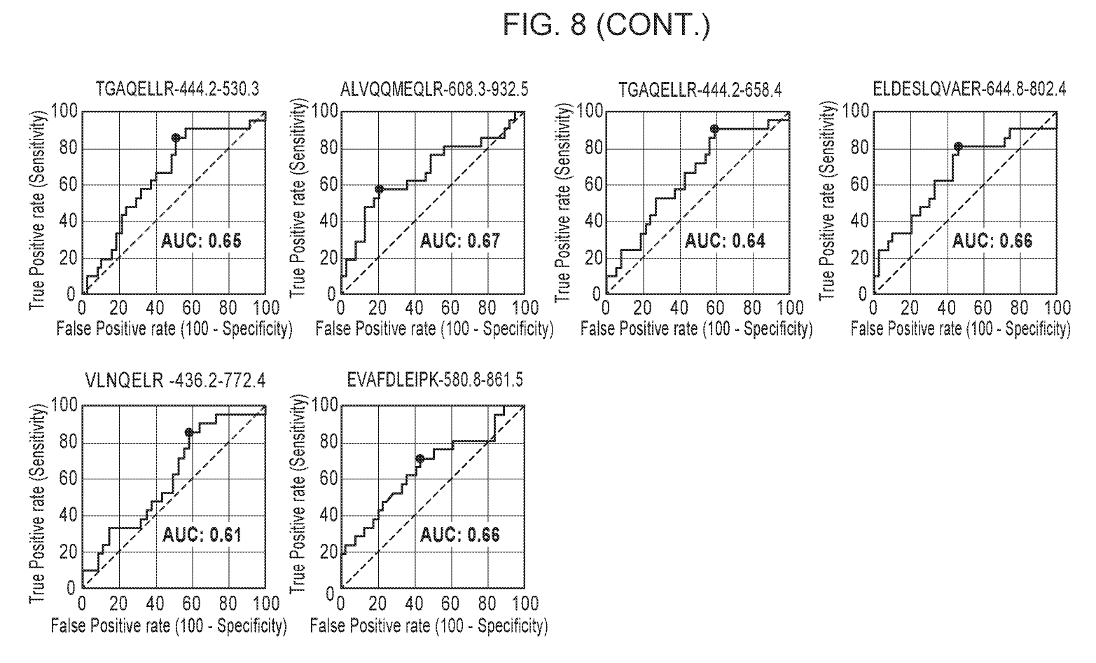

Next, a receiver operating characteristic (ROC) curve was generated for each significant transition to determine its individual diagnostic performance. The ROCs are shown in FIG. 8. Briefly, transition SEQ ID NO:1 (LIASMSSDSLR (590.3 1066.3)) had an AUC of 0.73, transition SEQ ID NO: 3 (IQNILTEEPK (592.8-829.4)) had an AUC of 0.72, transition SEQ ID NO: 1 (LIASMSSDSLR (590.3-953.2)) had an AUC of 0.71, transition SEQ ID NO: 3 (IQNILTEEPK (592.8 943.4)) had an AUC of 0.70, transition SEQ ID NO: 4 (GSLVQASEANLQAAQDFVR (1002.5-1448.6)) had an AUC of 0.66, transition SEQ ID NO: 5 (EIQNAVNGVK (536.3-417.2)) had an AUC of 0.66, transition SEQ ID NO: 6 (VLNQELR (436.2-659.3)) had an AUC of 0.63, transition SEQ ID NO: 4 (GSLVQASEANLQAAQDFVR (1002.5-1232.6)) had an AUC=0.64, transition SEQ ID NO: 7 (TGAQELLR (444.2-530.3)) had an AUC of 0.65, transition SEQ ID NO: 8 (ALVQQMEQLR (608.3-932.5)) had an AUC of 0.67, transition SEQ ID NO: 7 (TGAQELLR (444.2 658.4)) had an AUC of 0.64, transition SEQ ID NO: 9 (ELDESLQVAER (644.8-802.4)) had an AUC of 0.66, transition SEQ ID NO: 6 (VLNQELR (436.2-772.4)) had an AUC of 0.61 and transition SEQ ID NO: 10 (EVAFDLEIPK (580.8-861.5)) had an AUC of 0.66.

Each individual transition's performance showed modest diagnostic potential, the performance of all 7 proteins of the Alzheimer's disease panel was measured based on the combined performance of the 14 transitions. FIG. 9 shows the ROC for the 7-protein biomarker panel based on the combined performance of the 14 transitions. The AUC (AUC=0.82) based on a sensitivity of 67% and a specificity of 85%, showed an improved performance for the 7-protein biomarker panel as compared to any of the individual transition performances.

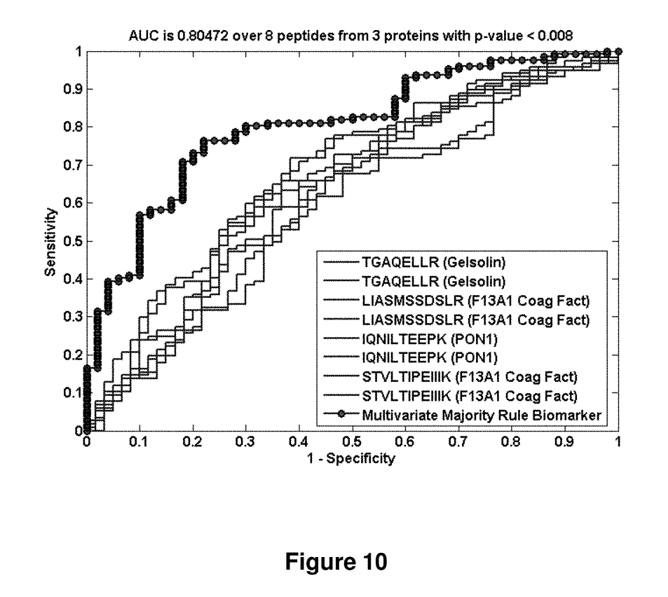

An additional ROC was generated for a 3-protein Alzheimer's disease panel (GSN, F13A1 and PON1) based on the combined performance of 8 transitions (see FIG. 10) representing 4 peptides (SEQ ID NO: 7 (TGAQELLR), SEQ ID NO: 1 (LIASMSSDSLR), SEQ ID NO: 3 (IQNILTEEPK), SEQ ID NO: 2 (STVLTIPEIIIK)). Like the combined performance of the 14 transitions discussed above, the combined performance of 8 transitions (AUC=0.80) was improved over the individual transition performances and the AUC. These results illustrate that the combined performance of the proteins and their transitions is greater than the sum of the individual markers.

What is claimed is:

1. A method for diagnosing Alzheimer's disease in a subject, comprising:
determining the protein expression of a plurality of proteins comprising at least F13A1, PON1, ITIH1, CLU, APOD, GSN, and APOA4 from a biological sample from the subject;
comparing the protein expression from step (a) to the protein expression of a plurality of proteins comprising at least F13A1, PON1, ITIH1, CLU, APOD, GSN, and APOA4 from a control biological sample, wherein the control biological sample is obtained from a subject with cognitive impairment due to the normal effects of aging or with no cognitive impairment;
diagnosing Alzheimer's disease in the subject based on the differential protein expression of the plurality of proteins between the subject biological sample and the control biological sample, wherein the subject is diagnosed with Alzheimer's disease if the differential protein expression has a statistical p value of 0.05 or below.

2. The method of claim 1, wherein diagnosing Alzheimer's disease occurs at least two years before the subject experiences the onset of mild to moderate cognitive impairment.

3. The method of claim 1, wherein diagnosing of Alzheimer's disease occurs at least four years before the subject experiences the onset of mild to moderate cognitive impairment.

4. The method of claim 1, further comprising administering a mini-mental state examination (MMSE) to the subject.

5. The method of claim 5, wherein diagnosing Alzheimer's disease in further comprises determining whether the subject has a MMSE score of less than 26.

6. The method of claim 6, wherein the subject has an MMSE score of less than 21.

7. The method of claim 7, wherein the subject has an MMSE score of less than 10.

8. The method of claim 1, wherein the biological sample is blood, plasma or a serum sample.

9. The method of claim 1, wherein protein expression can be determined by reverse transcriptase-polymerase chain reaction (RT-PCR), microarray, serial analysis of gene expression (SAGE), gene expression analysis by massively parallel signature sequencing (MPSS), immunoassays, immunohistochemistry (IHC), mass spectrometry (MS), transcriptomics, or proteomics.

10. The method of claim 10, wherein the mass spectrometry is chromatography-mass spectrometry (LC-MS) using eXtracted Ion Chromatograms (XIC), selected ion monitoring (SIM), selected reaction monitoring (SRM), multiple reaction monitoring mass spectrometry (MRM), or MRM-triggered MS/MS (MRM-MS/MS).

11. The method of claim 1, further comprising detecting one or more peptide transitions of the plurality of proteins, the peptide transitions comprising at least SEQ ID NO: 1 (LIASMSSDSLR (590.3-1066.3)), SEQ ID NO: 3 (IQNILTEEPK (592.8-829.4)), SEQ ID NO: 1 (LIASMSSDSLR (590.3-953.2)), SEQ ID NO: 3 (IQNILTEEPK (592.8-943.4)), SEQ ID NO: 4 (GSLVQASEANLQAAQDFVR (1002.5-1448.6)), SEQ ID NO: 5 (EIQNAVNGVK (536.3-417.2)), SEQ ID NO: 6 (VLNQELR (436.2-659.3)), SEQ ID NO: 4 (GSLVQASEANLQAAQDFVR (1002.5-1232.6)), SEQ ID NO: 7 (TGAQELLR (444.2-530.3)), SEQ ID NO: 8 (ALVQQMEQLR (608.3-932.5)), SEQ ID NO: 7 (TGAQELLR (444.2-658.4)), SEQ ID NO: 9 (ELDESLQVAER (644.8-802.4)), SEQ ID NO: 6 (VLNQELR (436.2-772.4)), or SEQ ID NO: 10 (EVAFDLEIPK (580.8-861.5)).

12. The method of claim 1, wherein the plurality of proteins comprises at least A1BG, APOA4, APOD, ARSA, ATP2A2, BDNF, CACNB2, CALML3, CDH5, CLU, COL18A1, COL1A2, CPN1, CSF1R, EPB41, EPHA8, F13A1, GALR3, CG, GNAQ, GPR113, GRIN2A, GRN, GSN, HPX, INADL, ITIH1, ITIH2, Kng1, LAMB2, LRP8, LTBP1, MMP16, MPDZ, MTOR, NMB, NTRK2, PACSIN1, PARD3, PKDREJ, PON1, PTPRB, SEMG1, SERPINA3, SERPINA4, SERPINF1, SNCB, SYTL4, TMPRSS2 and VTN.

13. A kit comprising reagents for determining the protein expression of a plurality of proteins comprising at least F13A1, PON1, ITIH1, CLU, APOD, GSN, and APOA4 from a biological sample from a subject and instructions for performing the method of claim 1.

14. The kit of claim 14, comprising reagents for detecting one or more peptide transitions of the plurality of proteins.

15. A kit of claim 15, comprising reagents for detecting one or more peptide transitions of the plurality of proteins, the peptide transitions comprising at least SEQ ID NO: 1 (LIASMSSDSLR (590.3-1066.3)), SEQ ID NO: 3 (IQNILTEEPK (592.8-829.4)), SEQ ID NO: 1 (LIASMSSDSLR (590.3-953.2)), SEQ ID NO: 3 (IQNILTEEPK (592.8-943.4)), SEQ ID NO: 4 (GSLVQASEANLQAAQDFVR (1002.5-1448.6)), SEQ ID NO: 5 (EIQNAVNGVK (536.3-417.2)), SEQ ID NO: 6 (VLNQELR (436.2-659.3)), SEQ ID NO: 4 (GSLVQASEANLQAAQDFVR (1002.5-1232.6)), SEQ ID NO: 7 (TGAQELLR (444.2-530.3)), SEQ ID NO: 8 (ALVQQMEQLR (608.3-932.5)), SEQ ID NO: 7 (TGAQELLR (444.2-658.4)), SEQ ID NO: 9 ELDESLQVAER (644.8-802.4), SEQ ID NO: 6 (VLNQELR (436.2-772.4)), or SEQ ID NO: 10 (EVAFDLEIPK (580.8-861.5)).


SEQUENCE LISTING (10 sequences)

SEQ ID NO: 1 - protein, 11 aa, Artificial Sequence
  Synthetic Polypeptide
LIASMSSDSLR

SEQ ID NO: 2 - protein, 12 aa, Artificial Sequence
  Synthetic Polypeptide
STVLTIPEIIIK

SEQ ID NO: 3 - protein, 10 aa, Artificial Sequence
  Synthetic Polypeptide
IQNILTEEPK

SEQ ID NO: 4 - protein, 19 aa, Artificial Sequence
  Synthetic Polypeptide
GSLVQASEANLQAAQDFVR

SEQ ID NO: 5 - protein, 10 aa, Artificial Sequence
  Synthetic Polypeptide
EIQNAVNGVK

SEQ ID NO: 6 - protein, 7 aa, Artificial Sequence
  Synthetic Polypeptide
VLNQELR

SEQ ID NO: 7 - protein, 8 aa, Artificial Sequence
  Synthetic Polypeptide
TGAQELLR

SEQ ID NO: 8 - protein, 10 aa, Artificial Sequence
  Synthetic Polypeptide
ALVQQMEQLR

SEQ ID NO: 9 - protein, 11 aa, Artificial Sequence
  Synthetic Polypeptide
ELDESLQVAER

SEQ ID NO: 10 - protein, 10 aa, Artificial Sequence
  Synthetic Polypeptide
EVAFDLEIPK